(19) United States
(12) Patent Application Publication
(10) Pub. No.: US 2014/0214043 A1
(43) Pub. Date: Jul. 31, 2014

(54) SURGICAL SPACER INSTRUMENT AND METHOD

(71) Applicant: WARSAW ORTHOPEDIC, INC., Warsaw, IN (US)

(72) Inventor: Roy Lim, Germantown, TN (US)

(73) Assignee: WARSAW ORTHOPEDIC, INC., Warsaw, IN (US)

(21) Appl. No.: 13/756,006

(22) Filed: Jan. 31, 2013

(51) Int. Cl. A61B 17/66

(52) U.S. Cl. CPC A61B 17/66
USPC 606/90

(57) ABSTRACT

A surgical instrument comprises a first member, an intermediate member and a second member. The intermediate member includes a first part and a second part. The second member includes a first arm and a second arm that extend from the first part. The arms are rotatable relative to the first part and engageable with the second part to axially translate the intermediate member for engagement with the first member to move the first member between a first orientation and a second orientation to space vertebral tissue. Systems and methods are disclosed.

### TECHNICAL FIELD

The present disclosure generally relates to medical devices for the treatment of spinal disorders, and more particularly to a surgical implant system and method for treatment of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. As part of these surgical treatments, implants, such as, for example, spinal constructs and interbody devices are often employed for stabilization of a treated section of a spine. For example, during surgical treatment, surgical instruments can be used to prepare a surgical site and the implants can be delivered to the surgical site for treating the spine section. This disclosure describes an improvement over these prior art technologies.

### SUMMARY

Accordingly, in one embodiment, in accordance with the principles of the present disclosure, a surgical instrument is provided. The surgical instrument comprises a first member, an intermediate member and a second member. The intermediate member includes a first part and a second part. The second member includes a first arm and a second arm that extend from the first part. The arms are rotatable relative to the first part and engageable with the second part to axially translate the intermediate member for engagement with the first member to move the first member between a first orientation and a second orientation to space vertebral tissue.

### BRIEF DESCRIPTION OF THE DRAWINGS

The present disclosure will become more readily apparent from the specific description accompanied by the following drawings, in which:

FIG. 1 is a side view of components of one embodiment of a spinal implant system in accordance with the principles of the present disclosure disposed with vertebrae;

FIG. 2 is a side view of the components of the system shown in FIG. 1 disposed with vertebrae;

FIG. 3 is a side view of the components of the system shown in FIG. 1 disposed with vertebrae;

FIG. 4 is a breakaway view of the components of the system shown in FIG. 1;

FIG. 5 is a breakaway view of components of the system shown in FIG. 1;

FIG. 6 is a perspective view of components of one embodiment of spinal implant system in accordance with the principles of the present disclosure;

FIG. 7 is a perspective view of the components of the system shown in FIG. 6;

FIG. 8 is a breakaway view of the components of the system shown in FIG. 6;

FIG. 9 is a perspective view of the components of the system shown in FIG. 6;

FIG. 10 is a breakaway view of the components of the system shown in FIG. 6;

FIG. 11 is a breakaway view of the components of the system shown in FIG. 6;

FIG. 12 is a breakaway view of the components of the system shown in FIG. 6;

FIG. 13 is a breakaway view of the components of the system shown in FIG. 6; and

FIG. 14 is a graphical representation diagram of force versus height for components of a spinal implant system in accordance with the principles of the present disclosure.

Like reference numerals indicate similar parts throughout the figures.

### DETAILED DESCRIPTION

The exemplary embodiments of the system and related methods of use disclosed are discussed in terms of medical devices for the treatment of musculoskeletal disorders and more particularly, in terms of a surgical implant system and method for treatment of a spine disorder.

In one embodiment, the present system includes a surgical instrument having a lever style opener that measures a distracted disc space and a corresponding distraction force value at the lever. In one embodiment, the system includes an instrument that measures the distracted disc height and a corresponding force unit to determine a distraction force in an intervertebral disc space using an empirically derived table.

In one embodiment, the instrument includes a lever style opener that allows for easier disc height measurement. In one embodiment, the instrument provides indicia of load and/or displacement readings that are obtained from the axial movement of a rod that translates to actuate a distal jack mechanism. In one embodiment, the instrument is optimized for a posterior surgical approach to the human body.

In one embodiment, the instrument includes a load displacement opener that has an increased opening capacity and a handle lock for accurate force readings. In one embodiment, the instrument includes handles that are compressed to a first orientation to provide a disc height measurement and additionally compressed to provide indicia of a force measurement.

In one embodiment, the system includes an instrument configured to provide for distraction height measurement and measurements for determining the force from one or more vertebral bodies exerted on an implant. In one embodiment, the instrument has a scissor jack tip. In one embodiment, a proximal end of the instrument is a lever style opening with a distraction height scale. In one embodiment, the instrument includes an upper cross bridge utilized for impaction. In one embodiment, the instrument includes levers that are compressed to distract disc space disposed at a distal end thereof to provide indicia of a height measurement. In one embodiment, the instrument includes a pointer to indicate measurement of disc space height. In one embodiment, the instrument measures disc height and determines a distraction force. In one embodiment, the instrument has a distractor tip that is configured to return to a closed position after expansion.

In one embodiment, the instrument includes a locking bar to lock a pointer in a fixed position to provide indicia of measurement. In one embodiment, the instrument includes a first scale for providing indicia of a measurement of a distraction height and a second scale for providing indicia of a measurement of a disc distraction force. In one embodiment, an empirically derived table is provided to determine the distraction force required to distract a disc space, for example, for a distraction height of 13 millimeters and 9 units of distraction force, 75 pounds of force is employed to distract the disc space.

In some embodiments, one or all of the components of the system may be disposable, peel pack and/or pre packed sterile devices. One or all of the components of the system may be reusable. The system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

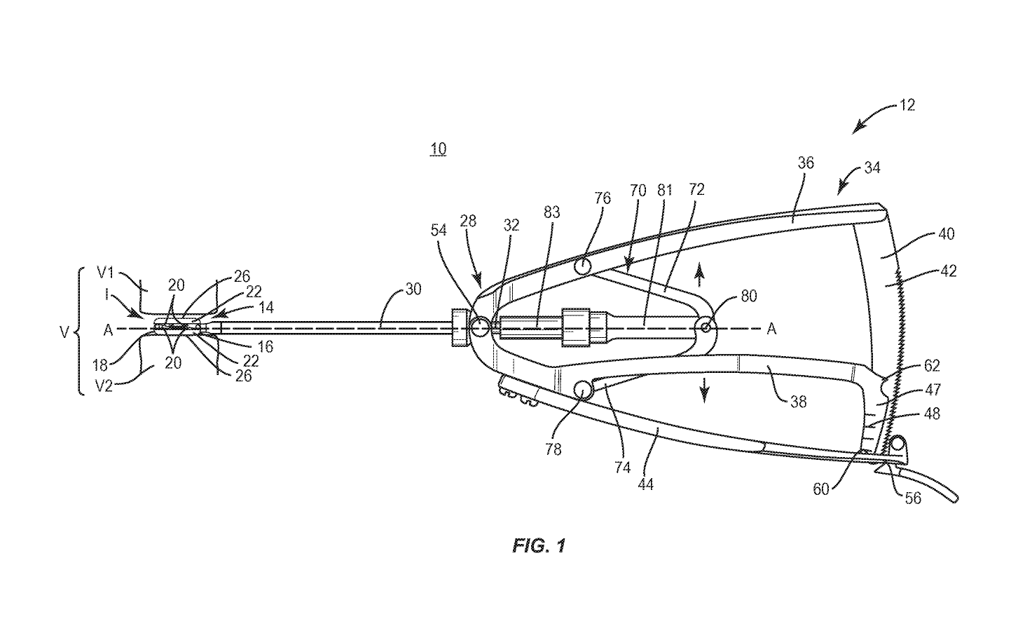

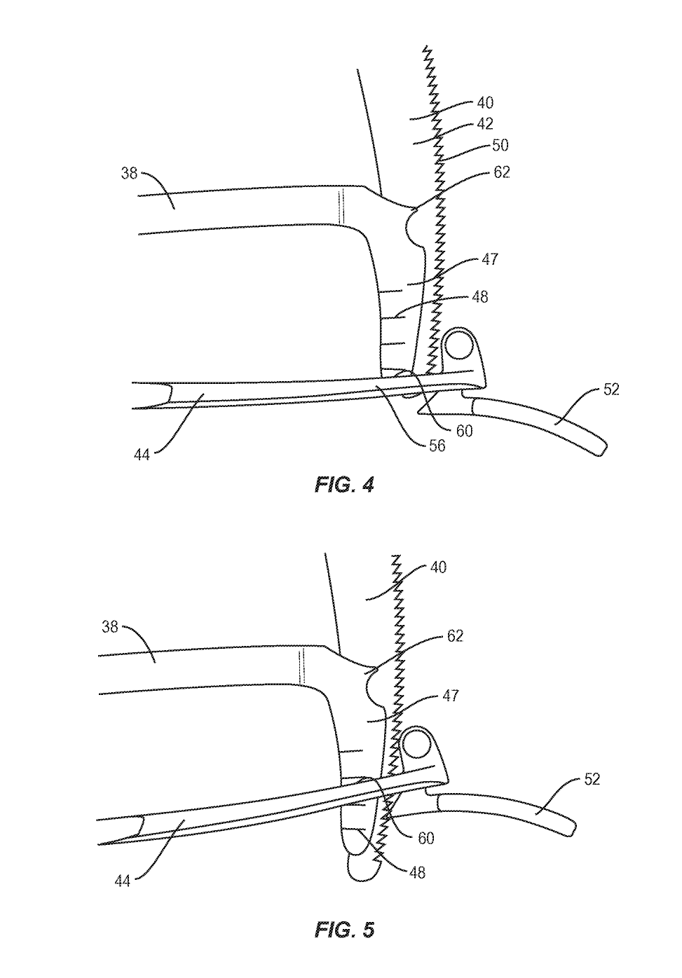

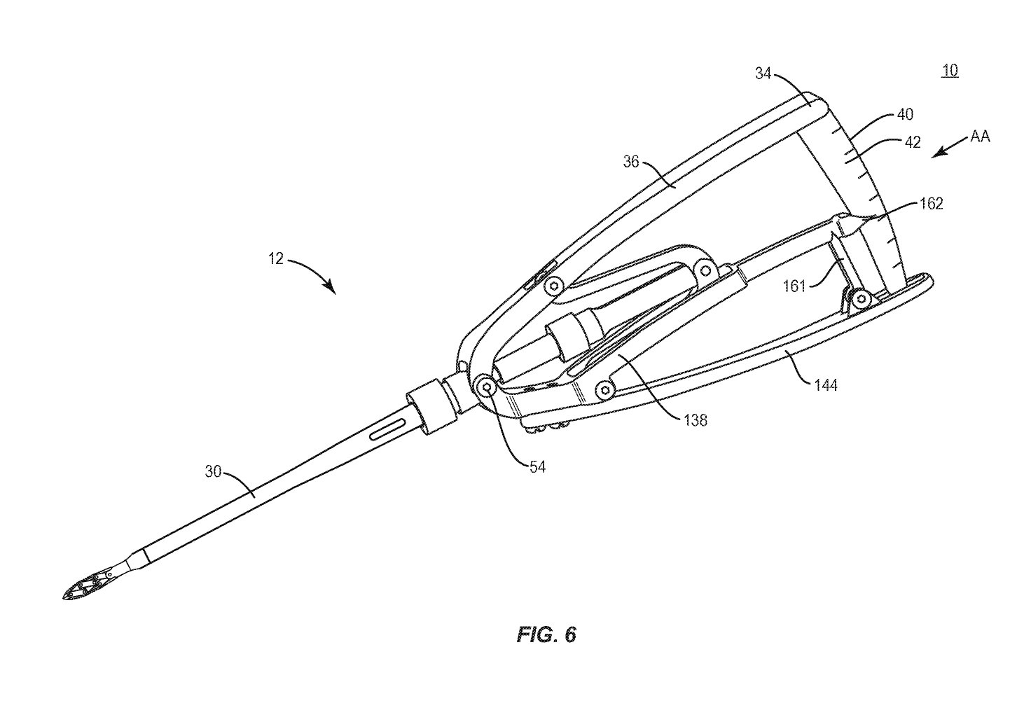

The following discussion includes a description of a surgical implant system, related components and methods of employing the system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a system, such as, for example, a spinal implant system 10 in accordance with the principles of the present disclosure.

The components of system 10 can be fabricated from biologically acceptable materials suitable for medical applications, including metals, synthetic polymers, ceramics, bone material, tissue and/or their composites, depending on the particular application and/or preference of a medical practitioner. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

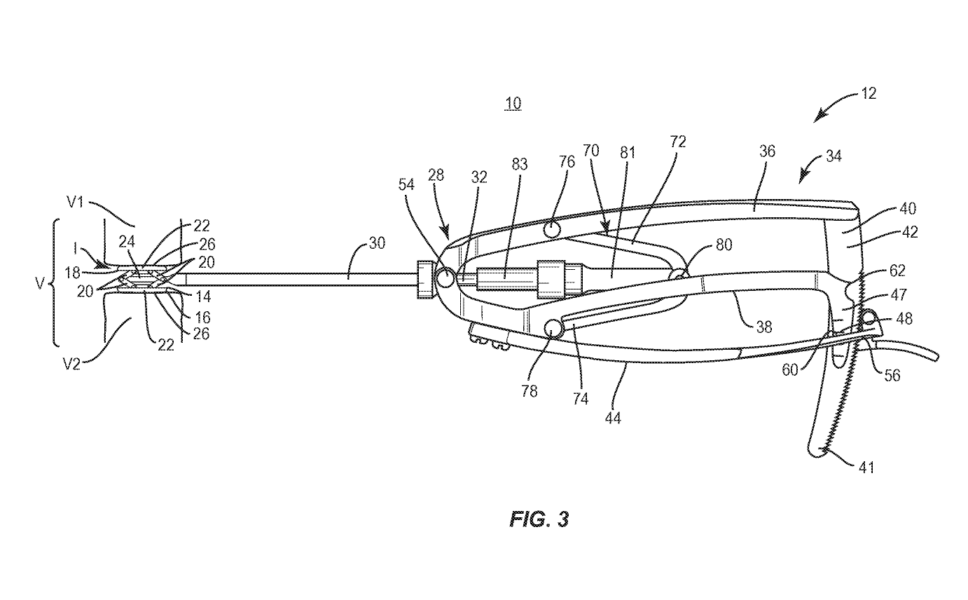

System 10 includes an instrument 12 configured for engagement with vertebrae. Instrument 12 includes a first member, such as, for example, a vertebral spacer 14. Spacer 14 includes a proximal end 16 and a distal end 18. Spacer 14 includes a linkage that may include a number of linkages 20 positioned between plates 22, depending upon the application. Each individual linkage 20 mates with a complimentary linkage 20 to provide movement to spacer 14. As shown in FIG. 3, spacer 14 includes two pairs of linkages 20 on a first side of a pull arm 24, and another two pairs of linkages 20 on a second side of arm 24 for a total of four pairs of linkages, or eight total linkages.

Each linkage 20 may have a variety of shapes and configurations. Plates 22 are positioned on a first side and a second side of spacer 14 to contact vertebral members, such as, for example, endplates of vertebrae. Plates 22 each include a contact surface 26 having a surface area to distribute the disc space load created by spacer 14 across a region of the vertebral members.

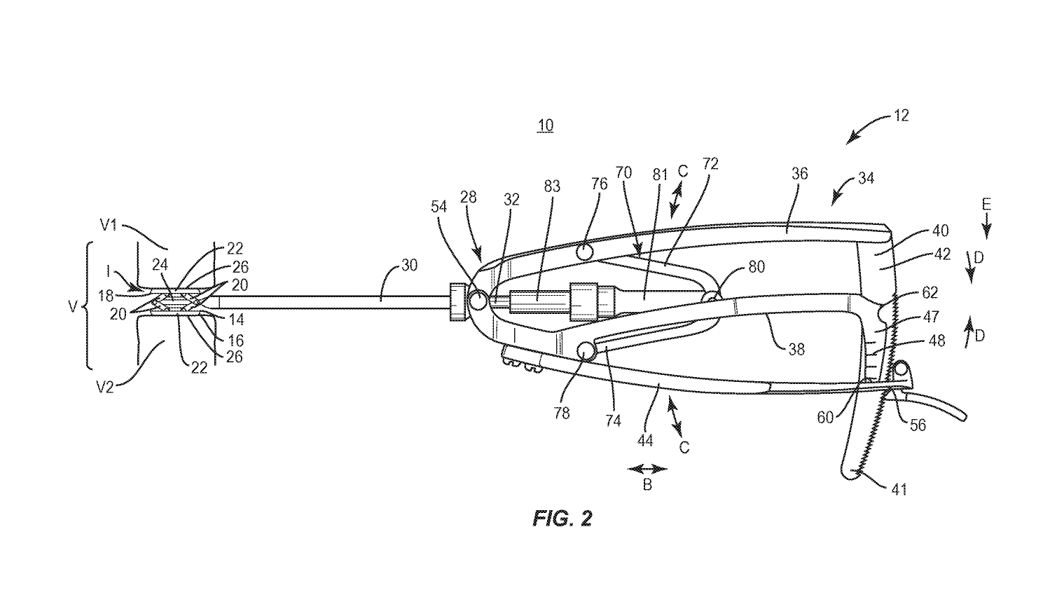

Spacer 14 is selectively adjustable between a first orientation, as shown in FIG. 1, and a second orientation, as shown in FIGS. 2 and 3. In the first orientation, such as, for example, a collapsed orientation, spacer 14 has a reduced size to facilitate introduction, insertion and delivery with a patient and/or a surgical pathway to a surgical site, and between vertebral members. In the second orientation, such as, for example, an expanded orientation, spacer 14 has an enlarged size for contacting, spacing apart and spreading the vertebral members. An arm (not shown) operatively connects the intermediate member, described herein, to linkages 20 to adjust spacer 14 to positions between a first orientation and a second orientation.

Instrument 12 includes an intermediate member, such as, for example, an actuator 28 configured to actuate spacer 14 between the first orientation and the second orientation. Actuator 28 extends from proximal end 16 of spacer 14 and defines a longitudinal axis A. Actuator 28 includes a part, such as, for example an outer sleeve 30 and a part, such as, for example, an inner shaft 32. Shaft 32 is configured to axially translate along axis A relative to sleeve 30. Sleeve 30 and shaft 32 each have a cylindrical cross section configuration. In some embodiments, sleeve 30 and/or shaft 32 may have alternate cross section shapes, such as, for example oval, oblong, triangular, square, polygonal, irregular, uniform, non-uniform, offset, staggered, undulating, arcuate, variable and/or tapered depending on a particular application.

Instrument 12 includes a second member, such as, for example, a lever 34. Lever 34 includes an arm 36 that extends from a collar of sleeve 30 and is rotatable about such connection. Arm 36 is pivotable about sleeve 30 between a first orientation and a second orientation of lever 34, described herein. Arm 36 is connected to sleeve 30 via a pivot, such as, for example, a hinge 54. Hinge 54 is centrally disposed adjacent sleeve 30 and configured to facilitate rotation of the components of arm 36 relative to axis A and about the connection of arm 36 and sleeve 30. In some embodiments, hinge 54 may be variously configured, such as, for example, pin, post, screw, living hinge, ratchet and/or concentric parts. In some embodiments, the cross section and/or overall configuration of arm 36 may be variously configured, such as, for example, round, oval, oblong, square, rectangular, polygonal, irregular, uniform, non-uniform, offset, staggered, tapered, consistent or variable, depending on the requirements of a particular application. In some embodiments, arm 36 may include an outer gripping surface configured for gripping by a hand of a practitioner. The gripping surface may be, such as, for example, rough, arcuate, undulating, mesh, porous, semi-porous, dimpled and/or textured according to the requirements of a particular application.

Arm 36 includes a transverse bridge 40 extending therefrom. Bridge 40 may be disposed at other angular orientations with arm 36, such as, for example, acute or obtuse, co-axial and/or may be offset or staggered with respect to arm 36. Bridge 40 is configured for movement through a cavity of extension 44, discussed herein. Bridge 40 includes visual indicia, such as, for example, graduations 42 indicating spacing dimension of the vertebral tissue.

Lever 34 includes a second arm 38 that extends from the collar of sleeve 30 and is pivotable between a first orientation and a second orientation of lever 34, described herein. Arm 38 is connected sleeve 30 via hinge 54. Hinge 54 is configured to facilitate rotation of the components of arm 38 relative to axis A. In some embodiments, the cross section and/or overall configuration of arm 38 may be variously configured, such as, for example, round, oval, oblong, square, rectangular, polygonal, irregular, uniform, non-uniform, offset, staggered, tapered, consistent or variable, depending on the requirements of a particular application. In some embodiments, that arm 38 may include an outer gripping surface configured for gripping by a hand of a practitioner. The gripping surface may be, such as, for example, rough, arcuate, undulating, mesh, porous, semi-porous, dimpled and/or textured according to the requirements of a particular application.

Arm 38 includes visual indicia, such as, for example, a pointer 62 that translates along bridge 40 such that pointer 62 is configured to indicate the measurement reading from indicia 42. Arm 38 includes a transverse extension 47. Extension 47 can be disposed at other angular orientations such as acute or obtuse, co-axial and/or may be offset or staggered with respect to arm 38. Extension 47 includes visual indicia, such as, for example, indicia 48. Indicia 48 indicates a distraction force required to distract vertebrae V1 and V2.

Arm 38 includes an extension 44 that extends in substantially parallel orientation to arm 38. In some embodiments, extension 44 can extend transverse and/or at other angular orientations such as acute or obtuse, co-axial and/or may be offset or staggered with respect to arm 38. Extension 44 is pivotably connected to arm 38. Extension 44 includes an end 56 having a surface 58 defining a cavity 60. In some embodiments, surface 56 may be rough, arcuate, undulating, mesh, porous, semi-porous, dimpled and/or textured according to the requirements of a particular application. Cavity 60 is configured to receive an end 41 of bridge 40 and arm 38 such that extension 44 provides a measurement reading, as discussed below.

Instrument 12 includes a linkage 70 configured to drive shaft 32 axially relative to sleeve 30, in the directions shown by arrows B in FIG. 2. Linkage 70 includes extensions 72 and 74. Extension 72 is pivotably connected to arm 36 via pivot 76. Extension 74 is pivotably connected to extension 46 via pivot 78. In some embodiments, pivot 76 and/or pivot 78 may be variously configured, such as, for example, pin, post, screw, living hinge, ratchet and/or concentric parts. In some embodiments, cross section and/or overall configuration of extension 72 and/or extension 74 may be variously configured, such as, for example, round, oval, oblong, square, rectangular, polygonal, irregular, uniform, non-uniform, offset, staggered, tapered, consistent or variable, depending on the requirements of a particular application. Extensions 72, 74 are pivotably connected to each other via a pivot 80. Pivot 80 is connected to a link 81 that is connected to shaft 32 at a collar 83. As lever 34 is actuated between a first collapsed orientation, as shown in FIG. 1, and a second expanded orientation, as shown in FIGS. 2 and 3, arms 36, 38 rotate and pivot about hinge 54 such that the components of linkage 70 rotate and pivot about pivots 76, 78, 80. As extensions 72, 74 pivot in opposing directions, as shown by arrows C in FIG. 2, extensions 72, 74 drive pivot 80, link 81 and collar 83 axially to drive shaft 32 in translation, in the directions shown by arrows B in FIG. 2. Translation of shaft 32 relative to sleeve 30 moves spacer 14 between collapsed orientations and expanded orientations in between vertebrae.

Arm 36 and arm 38 are movable between a first orientation and a second orientation. In the first orientation, arms 36, 38 are spaced apart in a non-compressed position, as shown in FIG. 1. In the second orientation, arms 36, 38 are disposed in a compressed position such that arms 36, 38 pivot about hinge 54 towards each other, as shown in FIGS. 2 and 3. Extension 47 is configured to be received by cavity 60 such that extension 44 translates along extension 47 to indicate the measurement of distraction force in the second orientation of arms 36, 38. When arms 36, 38 are disposed in the second orientation, extension 44 is positioned to indicate the force required to space the vertebral tissue. In the second orientation of arms 36, 38, extension 44 indicates a marking of spaced apart height of the vertebrae along extension 47. Pointer 62 identifies a marking of the distraction force required to space apart vertebrae as extension 46 translates along bridge 40.

Instrument 12 includes a lock to maintain instrument 12 in a fixed position to obtain accurate measurements. In one embodiment, as shown in FIG. 1, arm 36 includes a first lock surface such as, for example, a toothed rack 50 disposed along bridge 40. Arm 38 includes a second lock surface, such as, for example, a pivotable pawl 52 disposed at end 56. Toothed rack 50 and pivotable pawl 52 are engageable to fix spacer 14 and lever 34 in a selected orientation.

In operation, instrument 12 is manipulated to insert spacer 14 between vertebrae such that spacer 14 is disposed in a collapsed orientation, as shown in FIG. 1, to facilitate introduction, insertion and delivery of spacer 14 along a surgical pathway and/or at a surgical site. In the collapsed orientation, spacer 14 is disposed between vertebrae, engaging vertebral tissue and/or having one or both of plates 22 contacting vertebrae. Upon selective disposal of spacer 14 with vertebrae, arms 36, 38 of lever 34 are disposed in a non-compressed orientation, as shown in FIG. 1. Lever 34 is manipulated such that arms 36, 38 are compressed, as shown in FIG. 2, such that arms 36, 38 rotate about hinge 54, in the directions shown by arrows D. Compression of lever 34 causes linkage 70 to rotate and pivot about pivots 76, 78, 80 such that extensions 72, 74 pivot in opposing directions, as shown by arrows C. Extensions 72, 74 drive pivot 80, link 81 and collar 83 axially to drive shaft 32 in translation, in the directions shown by arrows B. Translation of shaft 32 relative to sleeve 30 actuates spacer 14 between the collapsed and expanded orientations in between vertebrae. As lever 34 is compressed, spacer 14 selectively expands to an enlarged size for contacting, spacing apart and/or spreading vertebrae. As arms 36, 38 are compressed, bridge 40 pivots towards arm 38, in the direction shown by arrow E. As bridge 40 pivots towards arm 38, an end of bridge 40 extends through cavity 60. As arm 38 is compressed, extension 44 translates along bridge 40 and extension 46 pivots towards arm 38. Arm 38 pivots through cavity 60.

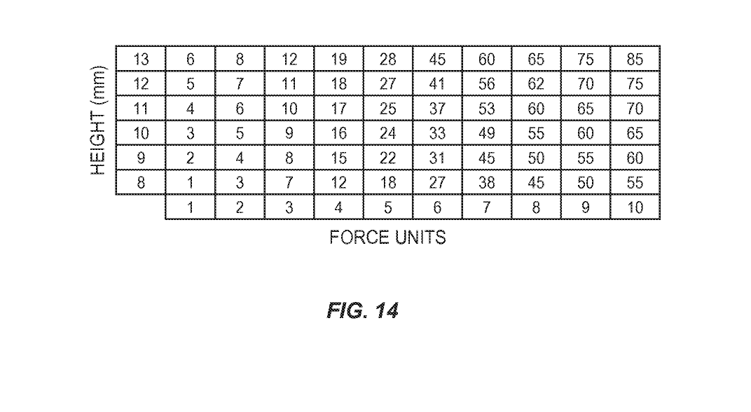

Upon selective adjustment of spacer 14 with vertebrae in an expanded orientation, instrument 12 measures a distracted disc space and a corresponding distraction force value for the vertebrae. In one embodiment, these measurements are employed to determine a distraction force for an intervertebral disc space using an empirically derived table, for example, as shown in FIG. 14. Compression of arms 36, 38 in a first compressed orientation of lever 34, as shown in FIG. 2, provides a displacement measurement, such as, for example, an intervertebral disc height measurement. Pointer 62 indicates measurement of disc space height via displacement readings 42 shown on bridge 40 corresponding to expansion of spacer 14 described herein. In the first compressed orientation of lever 34, instrument 12 can be locked by engaging toothed rack 50 and pawl 52.

Compression of arms 36, 38 in a second compressed orientation of lever 34, as shown in FIG. 3, provides a measurement of a disc distraction force, such as, for example, measurements for determining a force from one or more vertebrae exerted on an implant. Extension 44 indicates measurement of disc distraction force via distraction force readings 48 shown on extension 47 corresponding to force exerted on spacer 14 by vertebrae. In the second compressed orientation of lever 34, instrument 12 can be locked by engaging toothed rack 50 and pawl 52 to obtain accurate force readings.

For example, as shown in FIG. 14, an empirically derived table is provided to determine the distraction force required to distract a disc space, for example, for a distraction height of 13 mm and 9 units of distraction force, 75 pounds of force is employed to distract the disc space.

In assembly, operation and use, as shown in FIGS. 1-5, system 10, similar to that described herein, is employed with a surgical procedure, such as, for example, a fusion treatment of a spine of a patient including vertebrae V, intervertebral disc space I and body areas adjacent thereto, as discussed herein. In some embodiments, one or all of the components of system 10 can be delivered or implanted as a pre assembled device or can be assembled in situ. System 10 may be completely or partially revised, removed or replaced.

For example, system 10 can be employed with a surgical arthrodesis procedure, such as, for example, an interbody fusion for treatment of an applicable condition or injury of an affected section of a spinal column and adjacent areas within a body, such as, for example, intervertebral disc space I between a first vertebra V1 and second vertebra V2 of vertebrae V. In some embodiments, system 10 can include an intervertebral implant that can be inserted with intervertebral disc space I to space apart articular joint surfaces, provide support and maximize stabilization of vertebrae V. In some embodiments, system 10 may be employed with one or a plurality of vertebrae.

A medical practitioner obtains access to a surgical site including vertebrae V1, V2 in any appropriate manner, such as through incision and retraction of tissues. System 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. In one embodiment, the components of system 10 are delivered through a surgical pathway to the surgical site along a posterior surgical approach into intervertebral disc space I. Once access to the surgical site is obtained, the particular surgical procedure can be performed for treating the spine disorder.

An incision is made in the body of the patient and a cutting instrument (not shown) creates a surgical pathway for implantation of components of system 10. A preparation instrument (not shown) can be employed to prepare tissue surfaces of vertebrae V, as well as for aspiration and irrigation of a surgical region according to the requirements of a particular surgical application. Instrument 12 is manipulated to insert spacer 14 with disc space I and between vertebrae V1, V2 such that spacer 14 is disposed in a collapsed orientation, as shown in FIG. 1. Plate 22 is in contact with vertebra V2. Upon selective disposal of spacer 14 with vertebrae, arms 36, 38 of lever 34 are disposed in a non-compressed orientation, as shown in FIG. 1.

Lever 34 is compressed, as shown in FIG. 2, such that arms 36, 38 rotate about hinge 54, in the directions shown by arrows D Linkage 70 rotates and pivots about pivots 76, 78, 80 such that extensions 72, 74 pivot in opposing directions, as shown by arrows C. Extensions 72, 74 drive pivot 80, link 81 and collar 83 axially to drive shaft 32 in translation, in the directions shown by arrows B. Spacer 14 selectively expands to an enlarged size for contacting, spacing apart and/or spreading vertebrae V1, V2.

Bridge 40 pivots towards arm 38, in the direction shown by arrow E, such that an end of bridge 40 extends through cavity 60. Upon selective adjustment of spacer 14 with vertebrae in an expanded orientation, instrument 12 measures a distracted disc space and a corresponding distraction force value for the vertebrae. Compression of lever 34 in a first compressed orientation, as shown in FIG. 2, provides an intervertebral disc height measurement. Pointer 62 indicates measurement of disc space height, for example, a distraction height of 13 mm, via displacement readings 42 shown on bridge 40 corresponding to expansion of spacer 14 described herein.

As arm 38 is compressed, extension 44 translates along bridge 40 and extension 46 pivots towards arm 38. Compression of lever 34 in a second compressed orientation, as shown in FIG. 3, provides a measurement of a disc distraction force. Extension 44 indicates measurement of disc distraction force, for example, 9 units of distraction force, via distraction force readings 48 shown on extension 47 corresponding to force exerted on spacer 14 by vertebrae V1, V2. Lever 34 is locked by engaging toothed rack 50 and pawl 52 to obtain accurate force readings.

In one embodiment, in connection with the readings of distraction height and distraction force provided by the configuration of instrument 12, 75 pounds of force is required to distract the disc space for a selected intervertebral implant, according to the empirically derived table, as shown in FIG. 14. The components of system 10 can be made of radiolucent materials such as polymers. Radiomarkers may be included for identification under x-ray, fluoroscopy, CT or other imaging techniques. In some embodiments, the use of microsurgical and image guided technologies may be employed to access, view and repair spinal deterioration or damage, with the aid of system 10. Upon completion of the procedure, the surgical instruments, assemblies and non-implant components of system 10 are removed from the surgical site and the incision is closed.

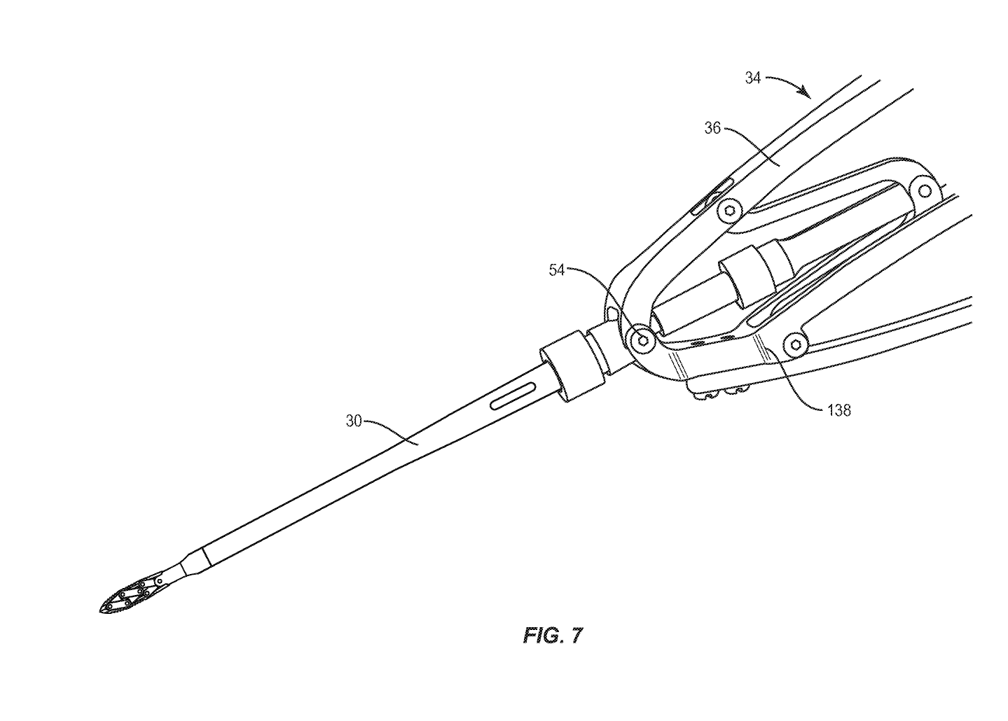

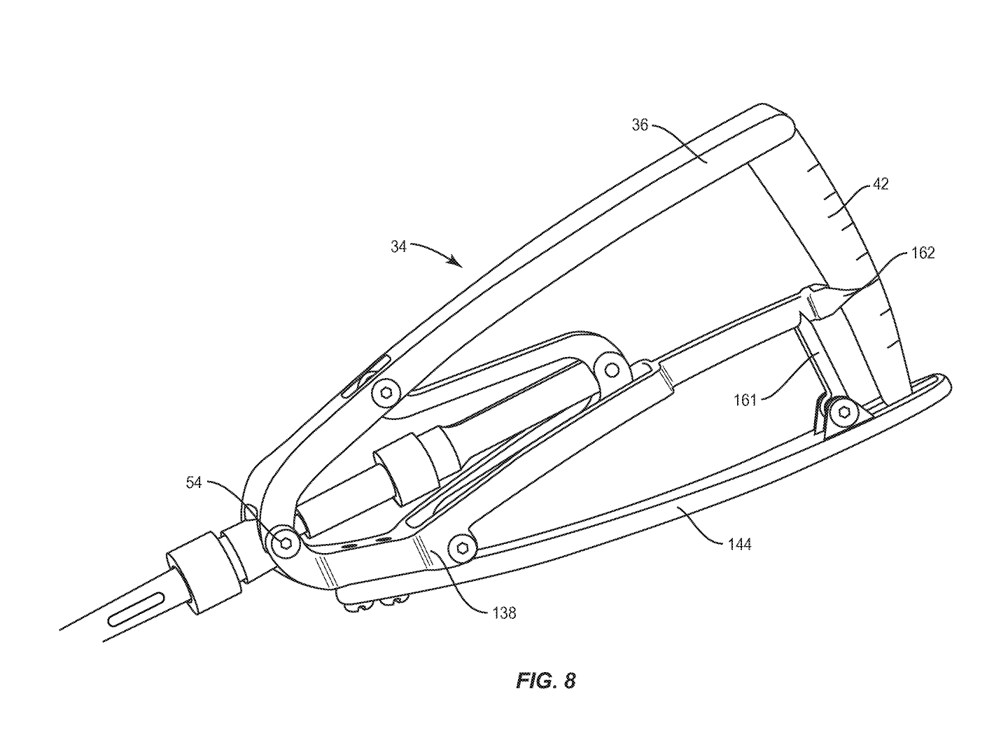

In one embodiment, as shown in FIGS. 6-13, system 10, similar to the systems and methods described herein, comprises instrument 12, which includes lever 34, described above. Lever 34 includes arm 36, described above, and an arm 138 that extends from the collar of sleeve 30 and is pivotable between a first orientation and a second orientation of lever 34, described herein. Arm 138 is connected sleeve 30 via hinge 54.

Arm 138 extends axially in a slight arcuate configuration and includes visual indicia, such as, for example, a pointer 162 that translates along bridge 40 such that pointer 162 is configured to indicate the measurement reading from indicia 42, similar to that described above. Bridge 40 includes an impact surface 147 that includes visual indicia, such as, for example, indicia 148. Indicia 148 indicates a distraction force required to distract vertebrae, similar to indicia 48 described above. In one embodiment, impact surface 147 is configured for receiving the force of a hammer or other similar device, for driving spacer 14 into a disc space and/or impacting components of system 10 with the disc space.

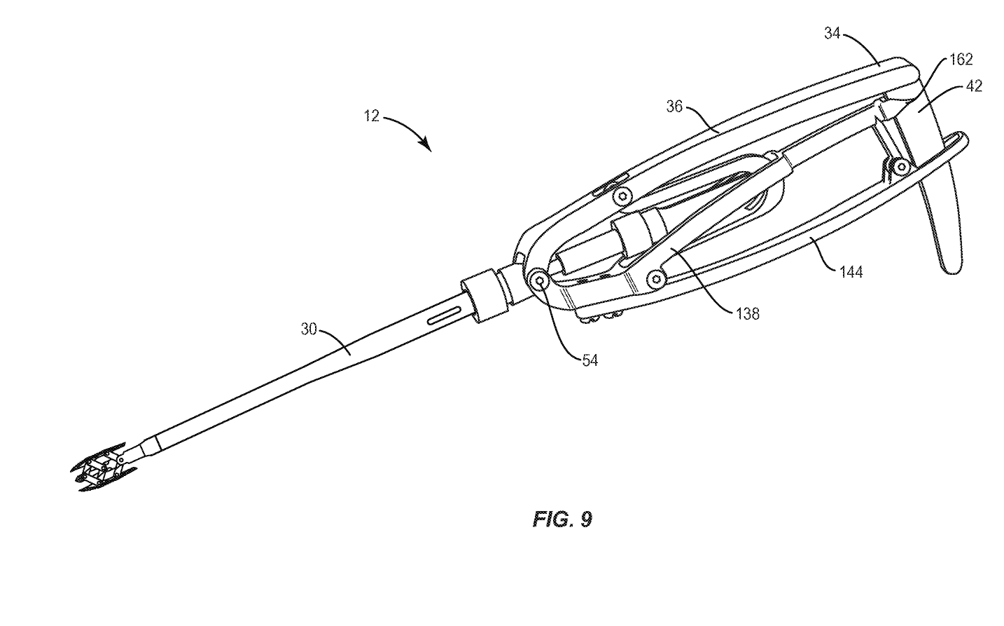

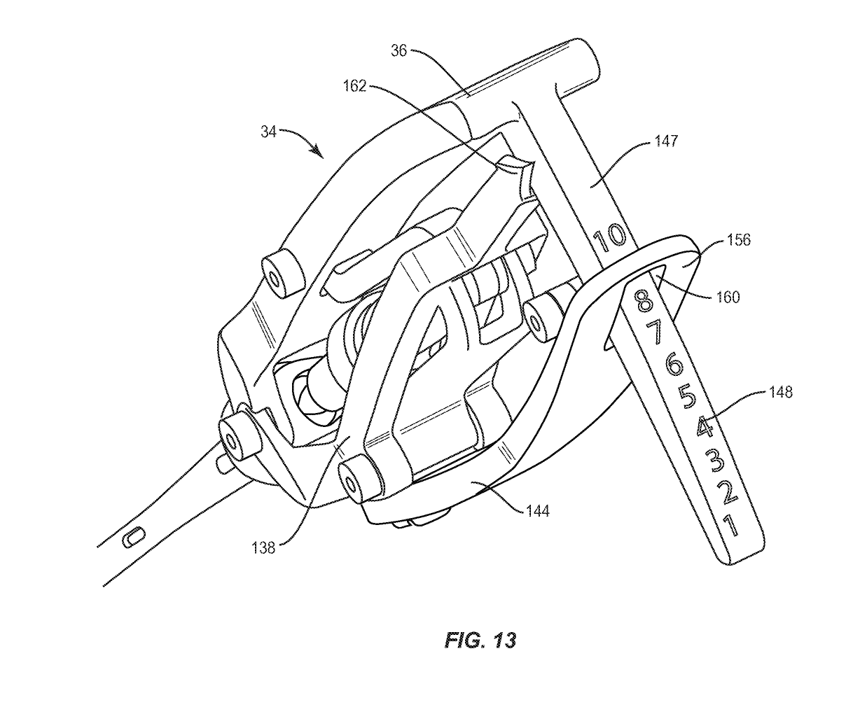

Arm 138 includes an extension 144 that extends in substantially parallel orientation to arm 138. Extension 144 is pivotably connected to arm 138. Extension 144 includes an end 156 defining a cavity 160, similar to cavity 60 described above. Extension 144 includes a support 161 disposed adjacent end 156. Support 161 is pivotably connected to extension 144 for rotation about end 156. Support 161 can be rotated into engagement with arm 138, as shown in FIGS. 8-11, to fix position of arm 138 relative to extension 144. Support 161 can be rotated out of engagement with arm 138, as shown in FIGS. 12-13, to facilitate movement of arm 138 relative to extension 144.

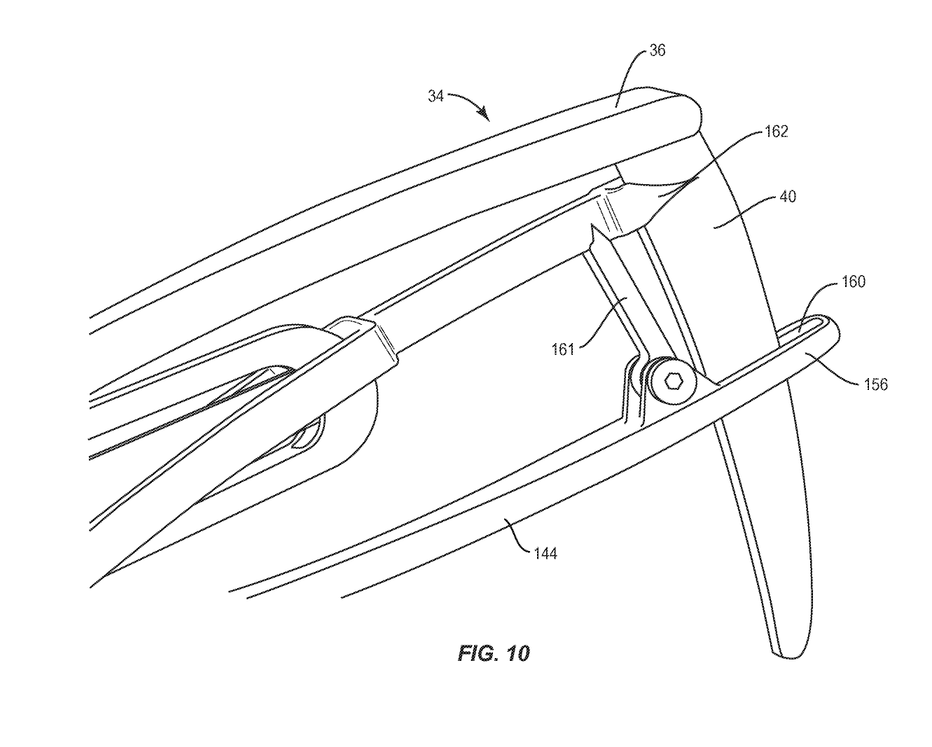

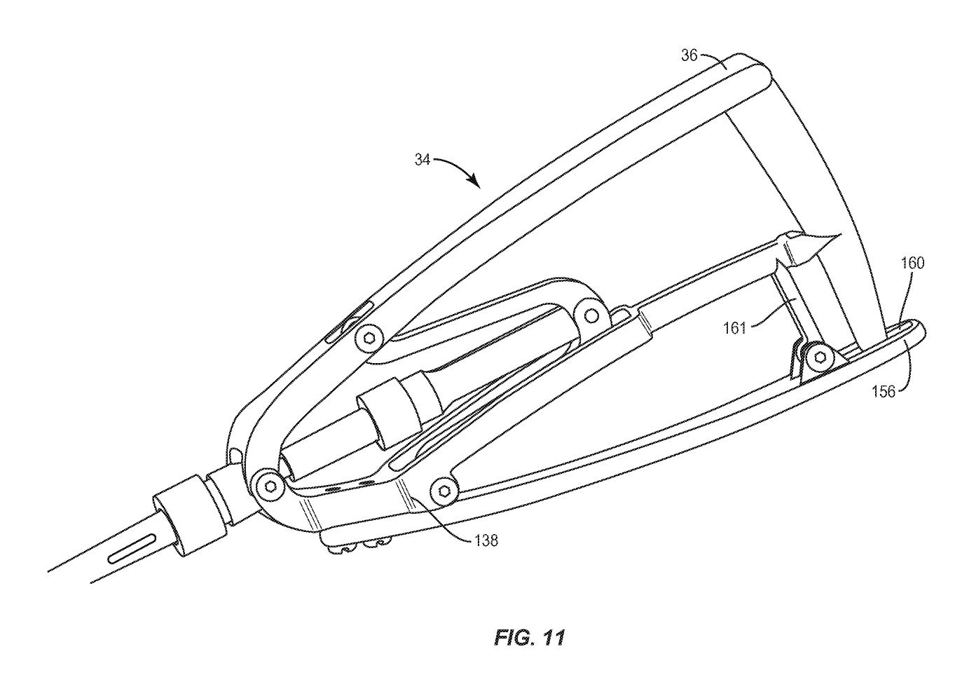

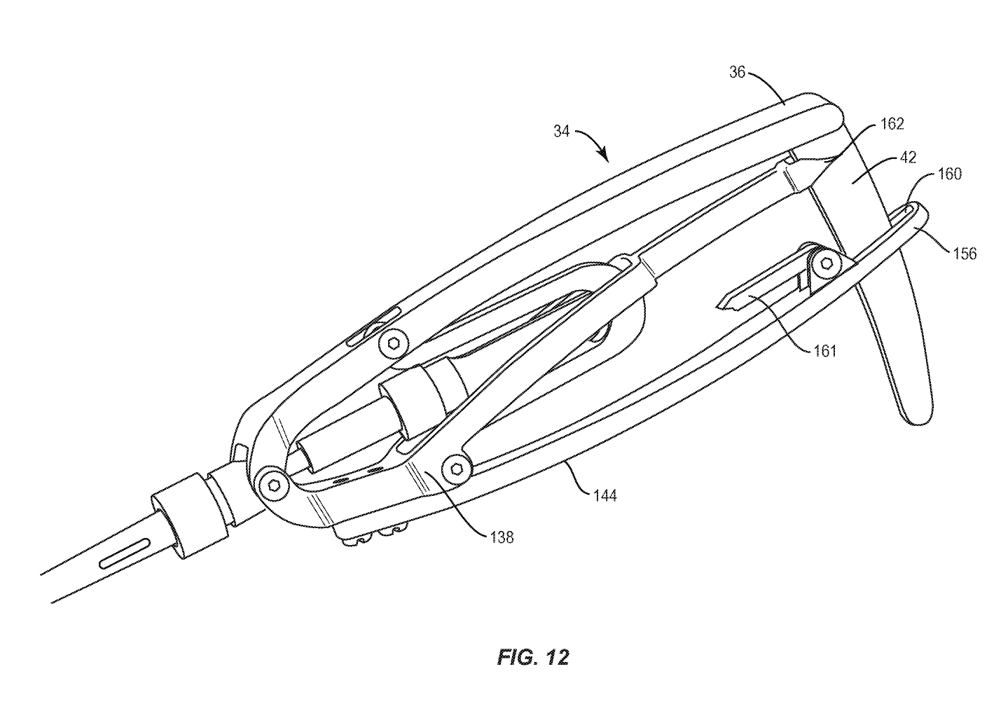

In operation, similar to operation of instrument 12 described above, surface 147 is impacted, as shown by arrow AA in FIG. 6, to insert spacer 14 into a disc space. Squeezing lever 34 distracts the disc space and results in a height measurement, as shown in FIG. 9, similar to that described above. Pointer 162 is used to determine the disc space height, as shown in FIG. 10. After the disc height has been determined, a distraction force can be estimated and spacer 14 is returned to a collapsed orientation, as shown in FIGS. 8 and 11.

Support 161 is released from pointer 162 and lever 34 is squeezed until pointer 162 returns to the previous disc height reading, for example, 13 mm, as shown in FIG. 12. A second scale, such as, for example, indicia 148 is used to determine the disc distraction force such that extension 144 indicates the disc distraction force, for example, 9 units of force. In one embodiment, in connection with the readings of distraction height and distraction force provided by the configuration of instrument 12, 75 pounds of force is required to distract the disc space for a selected implant, according to the empirically derived table, as shown in FIG. 14.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first member;
an intermediate member including a first part and a second part; and
a second member including a first arm and a second arm, the arms extending from the first part;
wherein the arms are rotatable relative to the first part and engageable with the second part to axially translate the intermediate member for engagement with the first member to move the first member between a first orientation and a second orientation to space vertebral tissue.

2. A surgical instrument as recited in claim 1, wherein the first part includes an outer sleeve and the second part includes an inner shaft that axially translates relative to the outer sleeve to engage the first member.

3. A surgical instrument as recited in claim 1, further comprising an arm linkage connecting the arms to the second part, the arm linkage being rotatable relative to the arms.

4. A surgical instrument as recited in claim 1, wherein the first arm includes a transverse extension configured for movement through a cavity of the second arm.

5. A surgical instrument as recited in claim 4, wherein the transverse extension includes indicia representing spacing dimensions of the vertebral tissue.

6. A surgical instrument as recited in claim 4, wherein the transverse extension includes indicia representing a distraction force to space the vertebral tissue.

7. A surgical instrument as recited in claim 1, wherein the first arm includes indicia of dimension and in the second orientation, the second arm is disposed to indicate a dimension of the spaced vertebral tissue.

8. A surgical instrument as recited in claim 1, wherein the first arm includes indicia of distraction force and in the second orientation, the second arm is disposed to indicate a force required to space the vertebral tissue.

9. A surgical instrument as recited in claim 1, wherein the first arm includes indicia of dimension and the second arm includes a first extension and a second extension having indicia of distraction force such that in the second orientation, the second extension is disposed to indicate a dimension of the spaced vertebral tissue and the first extension is disposed to indicate a force required to space the vertebral tissue.

10. A surgical instrument as recited in claim 1, wherein the first arm includes a first indicia of dimension and a second indicia of distraction force and the second arm includes a first extension and a second extension such that in the second orientation, the second extension is disposed to indicate a dimension of the spaced vertebral tissue and the first extension is disposed to indicate a force required to space the vertebral tissue.

11. A surgical instrument as recited in claim 1, wherein the first arm includes a first lock surface and the second arm includes a second lock surface, the lock surfaces being engageable to fix the first member in a selected orientation.

12. A surgical instrument as recited in claim 11, wherein the first lock surface includes a toothed rack and the second lock surface includes a pivotable pawl such that the first member is releasable from the selected orientation.

13. A surgical instrument as recited in claim 1, wherein the first member includes a first surface configured to engage a first vertebral member and a second surface configured to engage a second vertebral member, the first member being movable to space apart the vertebral members at a selected height and at a selected distraction force.

14. A surgical instrument as recited in claim 1, wherein the first member includes an expandable linkage.

15. A surgical instrument comprising:
a first member;
an intermediate member including a first part and a second part;
a second member including a first arm having indicia of dimension and a second arm having indicia of distraction force, the arms extending from the first part,
wherein the arms are rotatable relative to the first part and engageable with the second part to axially translate the intermediate member for engagement with the first member to move the first member between a collapsed orientation and an expandable orientation to space vertebrae such that the second arm is disposed to indicate a dimension of the spaced vertebral tissue and a distraction force required to space the vertebral tissue.

16. A surgical instrument as recited in claim 15, wherein the first arm includes a transverse extension having the indicia of dimension.

17. A surgical instrument as recited in claim 16, wherein the transverse extension includes an impact surface.

18. A surgical instrument as recited in claim 15, wherein the second arm includes a first extension and a second extension having the indicia of distraction force such that in the second orientation, the first extension is disposed to indicate the distraction force.

19. A surgical instrument as recited in claim 15, wherein the first arm includes a first lock surface and the second arm includes a second lock surface, the lock surfaces being engageble to fix the first member in a selected orientation.

20. A vertebral distractor comprising:
a vertebral spacer including an expandable linkage;
an actuator including an outer sleeve and an inner shaft configured for movement with the sleeve; and
a lever including a first arm having a bridge that displays graduated markings of height and a second arm having a first extension and a second extension that displays graduated markings of distraction force, the arms extending from the sleeve and being pivotally movable relative thereto,
wherein the arms are pivotable about the sleeve and engageable with the shaft to axially translate the actuator into engagement with the linkage to move the spacer between a collapsed orientation and an expanded orientation to space a first vertebra apart from a second vertebra such that the second extension identifies a marking of spaced apart height of the vertebrae and the first extension identifies a marking of the force required to space apart vertebrae.
